(19) United States
(12) Patent Application Publication
(10) Pub. No.: US 2025/0360092 A1
(43) Pub. Date: Nov. 27, 2025

(54) Carotenoid-fortified Yogurt and Methods For Preparing The Same

(71) Applicant: Maravilla, LLC, Birmingham, MI (US)

(72) Inventors: Alfonzo PRADO-CABRERO, Waterford (IE); John Nolan, Waterford (IE); Hao Shi, Wuhan (CN)

(21) Appl. No.: 19/215,596

(22) Filed: May 22, 2025

Related U.S. Application Data
(60) Provisional application No. 63/651,171, filed on May 23, 2024.
(60) Provisional application No. 63/687,589, filed on Aug. 27, 2024.

(51) Int. Cl. A61K 31/047; A23C 9/13

(52) U.S. Cl. CPC A61K 31/047; A23C 9/1307; A23C 2210/15

(57) ABSTRACT

A carotenoid-fortified yogurt includes greater than or equal to about 0.001 weight percent to less than or equal to about 0.03 weight percent of one or more carotenoids. A method for preparing the carotenoid-fortified yogurt includes contacting the one or more one or more carotenoids and a milk to prepare a mixture, heating the mixture to a temperature greater than or equal to about 4° C. to less than or equal to about 20° C., pressurizing the heated mixture at a first pressure greater than or equal to about 150 bar to less than or equal to about 250 bar, and after the pressurizing of the mixture at the first pressure, pressurizing the heated mixture at a second pressure greater than or equal to about 40 bar to less than or equal to about 60 bar.

### CROSS-REFERENCE TO RELATED APPLICATIONS

This application claims the benefit of U.S. Provisional Application No. 63/651,171 filed on May 23, 2024 and U.S. Provisional Application No. 63/687,589 filed Aug. 27, 2024. The entire disclosures of the above applications are incorporated herein by reference.

### FIELD

The present disclosure relates to carotenoid-fortified yogurt and methods for preparing the same.

### BACKGROUND

This section provides background information related to the present disclosure which is not necessarily prior art.

The carotenoid lutein is widely present in plants and animals, where it plays crucial physiological roles. In addition to providing the recognizable yellow hue to flowers and fruits, lutein helps plants dissipate excess energy under intense light conditions through its photoprotective and antioxidant properties. In animals, lutein prevents phototoxicity in the eye and plays a role in immunomodulation, oxidative status, and feather pigmentation. However, since animals are unable to synthesize lutein themselves, they must obtain this carotenoid from their diet.

In humans, the best-known beneficial effect of lutein is related to vision and cognitive function. This carotenoid, along with its structural isomer zeaxanthin, traverses the blood-brain barrier and accumulates in brain regions such as the hippocampus or the prefrontal, frontal, auditory, and occipital cortices, accounting for about 60 percent of total carotenoids found in these brain regions. In the retina, lutein, together with zeaxanthin and meso-zeaxanthin, accumulates in the macula, where it protects the cone photoreceptors from harmful blue light and reactive oxygen species. Likely as a consequence of these roles in the retina, lutein contributes to reducing the likelihood of progression of age-related macular degeneration and enhancing vision by improving contrast sensitivity. Although the mechanisms by which lutein improves cognitive function are not fully understood, studies indicate that lutein plays a crucial role in inhibiting DHA oxidation, stimulating the differentiation of human stem cells to neural progenitor cells, maintaining membrane rigidity and stability, and support communication between neurons. At the clinical level, lutein supplementation has been shown to enhance learning performance in children and improve cognitive function in the elderly. Interestingly, low plasma levels of lutein in human trials have been associated with an increased risk of Alzheimer's disease, while lutein, supplemented with zeaxanthin, meso-zeaxanthin and omega-3 fatty acids, has been shown to improve mood in Alzheimer patients. Recent research further indicates that lutein also plays a role in maintaining cardiovascular health, with recent results reporting that lutein, supplemented with the other macular carotenoids, lowers the levels of the circulating pro-inflammatory cytokines IL-6, IL-1β, TNF-α, and the levels of oxidized LDL in humans. Other roles of lutein in human health are preventing skin aging and enhancing bone density.

Despite the widespread presence of lutein in green leafy vegetables such as spinach, kale, and broccoli, as well as in egg yolk and animal fats, the average intake of lutein by the general population is only around 2 milligrams per day, which is far below the recommended daily intake by researchers and some governments. While dietary supplements offer a means to increase lutein intake, this form of increasing lutein ingestion might not be suitable for children and elderly, who may have swallowing difficulties, known as dysphagia. As an additional source for supplementing lutein intake, researchers have been striving to develop lutein-fortified foods, including milk, yoghurt, cheese, bread, muffins, oils, and beverages. However, no commercially available lutein-fortified food has been available so far.

### SUMMARY

This section provides a general summary of the disclosure and is not a comprehensive disclosure of its full scope or all of its features.

At least one example embodiment relates to a carotenoid-fortified yogurt.

In at least one example embodiment, the carotenoid-fortified yogurt may include greater than or equal to about 0.001 weight percent to less than or equal to about 0.03 weight percent of one or more carotenoids.

In at least one example embodiment, the one or more carotenoids may include lutein, zeaxanthin, astaxanthin, lycopene, or any combination thereof.

In at least one example embodiment, the one or more carotenoids may include lutein.

In at least one example embodiment, the carotenoid-fortified yogurt may include about 0.5 weight precent of the one or more carotenoids.

In at least one example embodiment, the carotenoid-fortified yogurt may include greater than or equal to about 1 milligrams to less than or equal to about 30 milligrams of the one or more carotenoids per 100 grams of yogurt.

In at least one example embodiment, the carotenoid-fortified yogurt may include about 15 milligrams of the one or more carotenoids per 100 grams of yogurt.

In at least one example embodiment, the carotenoid-fortified yogurt may further include a mouthfeel flavor.

In at least one example embodiment, the mouthfeel flavor may have an expressed color similar to that of the one or more carotenoids.

In at least one example embodiment, the one or more carotenoids may include lutein and the mouthfeel flavor may include peach, banana, or a combination thereof.

At least one example embodiment relates to a method for preparing a carotenoid-fortified yogurt.

In at least one example embodiment, the method may include pressurizing a mixture including one or more carotenoids and milk at a first pressure greater than or equal to about 150 bar to less than or equal to about 250 bar, and after the pressurizing of the mixture at the first pressure, pressurizing the mixture at a second pressure greater than or equal to about 40 bar to less than or equal to about 60 bar.

In at least one example embodiment, the mixture may include greater than or equal to about 0.001 weight precent to less than or equal to about 0.03 weight percent of the one or more carotenoids.

In at least one example embodiment, the first pressure may be about 200 bar and the second pressure is about 50 bar.

In at least one example embodiment, the method may further include depressurizing the mixture from the first pressure to the second pressure.

In at least one example embodiment, the method may further include, before the pressurizing of the mixture at the first pressure, heating the mixture to a temperature greater than or equal to about 55° C. to less than or equal to about 75° C.

In at least one example embodiment, the temperature may be about 65° C.

In at least one example embodiment, the method may further include preparing the mixture by contacting the one or more carotenoids and milk at a temperature greater than or equal to about 4° C. to less than or equal to about 20° C.

At least one example embodiment relates to a method for preparing a carotenoid-fortified yogurt.

In at least one example embodiment, the method may include contacting one or more one or more carotenoids and a milk to prepare a mixture, heating the mixture to a temperature greater than or equal to about 4° C. to less than or equal to about 20° C., pressurizing the heated mixture at a first pressure greater than or equal to about 150 bar to less than or equal to about 250 bar, and after the pressurizing of the mixture at the first pressure, pressurizing the heated mixture at a second pressure greater than or equal to about 40 bar to less than or equal to about 60 bar.

In at least one example embodiment, the mixture may include greater than or equal to about 0.001 weight percent to less than or equal to about 0.03 weight percent of the one or more carotenoids.

In at least one example embodiment, the method may further include depressurizing the mixture from the first pressure to the second pressure.

In at least one example embodiment, the one or more carotenoids may include lutein.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flowchart illustrating an example method for preparing a carotenoid-fortified yogurt sample in accordance with at least one example embodiment of the present disclosure.

FIG. 2 is a graphical demonstration illustrating the concentrations of lutein and zeaxanthin in an example carotenoid-fortified yogurt sample as prepared in accordance with at least one example embodiment of the present disclosure and over a thirty-five-day period of time at 4° C.

FIG. 3A is a demonstration comparing the appearance, aroma, and mouthfeel attributes of a blank, non-carotenoid-fortified yogurt sample and a lutein-fortified yogurt sample as prepared in accordance with at least one example embodiment of the present disclosure.

FIG. 3B is a demonstration comparing the flavor and taste attributes of the blank, non-carotenoid-fortified yogurt sample and the lutein-fortified yogurt sample as prepared in accordance with the at least one example embodiment of the present disclosure.

FIG. 4A is a high-performance liquid chromatography carotenoid profile for the blank, non-carotenoid-fortified yogurt sample.

FIG. 4B is a high-performance liquid chromatography carotenoid profile for a lutein-fortified yogurt sample as prepared in accordance with at least one example embodiment of the present disclosure without saponified treatment.

FIG. 4C is a high-performance liquid chromatography carotenoid profile for the lutein-fortified yogurt sample as prepared in accordance with at least one example embodiment of the present disclosure with saponification treatment.

FIG. 5 is a graphical demonstration illustrating the concentrations of lutein, a-cryptoxanthin, and zeaxanthin in serum collected from participants in a human bioavailability study, where the participants consumed a lutein-fortified yogurt sample as prepared in accordance with at least one example embodiment of the present disclosure.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected, or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein do not imply a sequence or order unless clearly indicated by the context. Thus, a first step, element, component, region, layer, or section discussed below could be termed a second step, element, component, region, layer, or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates both exactly or precisely the stated numerical value, and also, that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%. In addition, disclosure of ranges includes disclosure of all values and further divided ranges within the entire range, including endpoints and sub-ranges given for the ranges.

Example embodiments will now be described more fully with reference to the accompanying drawings.

Dairy products have historically been a vital component of the human diet, offering high-quality protein such as casein and whey, along with essential minerals like calcium, potassium, and phosphorus. In recent years, growing interest has focused on dairy products as carriers of functional ingredients, with yogurt emerging as a notable candidate. Yogurt is widely accepted as a healthy food and is especially popular among lactose-intolerant people. The presence of abundant amphiphilic proteins in yogurt, along with its semi-solid nature, contributes to the stability of fat-soluble bioactives, such as lutein, in the form of stable micelles. Furthermore, compared to other dairy products, yogurt offers wide flexibility in color and flavor modification and is also popular among children. Yogurt is also considered a suitable food for individuals with dysphagia due to its appropriate texture and moderate consistency. Typically falling within the 4 to 7 range on the International Dysphagia Diet Standardization Initiative scale, yogurt is recommended in clinical practice as a training meal for patients with swallowing difficulties.

Yogurt fortified with carotenoids and methods for preparing the same are provided. For example, in various aspects, the present disclosure provides a carotenoid-fortified yogurt that includes one or more carotenoids. The one or more carotenoids may include lutein, zeaxanthin, astaxanthin, lycopene, or any combination thereof. For example, in at least one example embodiment, the carotenoid-fortified yogurt may include lutein as a first carotenoid and a second carotenoid, where the second carotenoid includes zeaxanthin, astaxanthin, lycopene, or any combination thereof.

In at least one example embodiment, the carotenoid-fortified yogurt may include greater than or equal to about 0.001 weight percent to less than or equal to about 0.03 weight percent of the one or more carotenoids. For example, the lutein-fortified yogurt may include about 0.015 weight percent of the one or more carotenoids. In at least one example embodiment, the carotenoid-fortified yogurt may include greater than or equal to about 0.5 milligrams to less than or equal to about 40 milligrams (e.g., greater than or equal to about 1 milligrams to less than or equal to about 30 milligrams) of the one or more carotenoids per 100 grams of yogurt. For example, the lutein-fortified yogurt may include about 15 milligrams of the one or more carotenoids per 100 grams of yogurt.

In at least one example embodiment, the carotenoid-fortified yogurt may include greater than or equal to about 0.001 weight percent to less than or equal to about 0.03 weight percent of lutein. For example, the carotenoid-fortified yogurt may include about 0.015 weight percent of lutein. The carotenoid-fortified yogurt may include greater than or equal to about 0.5 milligrams to less than or equal to about 40 milligrams (e.g., greater than or equal to about 1 milligrams to less than or equal to about 30 milligrams) of lutein per 100 grams of yogurt. For example, in at least one example embodiment, the lutein-fortified yogurt may include about 15 milligrams of lutein per 100 grams of yogurt.

The one or more carotenoids may be provided as a carotenoid formulation (e.g., lutein formulation) that is dispersible in an aqueous solution and that includes a standard and an emulsifier. The carotenoid formulation should be water dispersible because its first contact with milk to prepare the carotenoid-fortified yogurt is with the water-phase of the milk. It is only later, as detailed below in the instance of the pressure treatment, that the carotenoid is embedded within fat globules of the milk. In at least one example embodiment, the one or more carotenoids may be extracted from marigold flowers (Tagetes erecta) and standardized with sunflower oil and emulsified using a food grade emulsifier. In at least one example embodiment, the carotenoid formulation may include a lutein formulation and the lutein formulation may include Plantcol Lutein Extract WS3.

In at least one example embodiment, the carotenoid-fortified yogurt may further include a mouthfeel flavor. For example, the lutein-fortified yogurt may include greater than or equal to about 2 weight percent to less than or equal to about 8 weight percent (e.g., about 6 weight percent) of the mouthfeel flavor. The mouthfeel flavor may be selected to include a fruit whose color is similar to that of the carotenoid. For example, when the one or more carotenoids include lutein, the mouthfeel flavor may include peach, banana, or a combination thereof.

In each instance, the carotenoid-fortified yogurt is stable over the entire shelf life of the product and the one or more carotenoids (e.g., lutein) is efficiently absorbed by the human digestive system, as further detailed in the examples below.

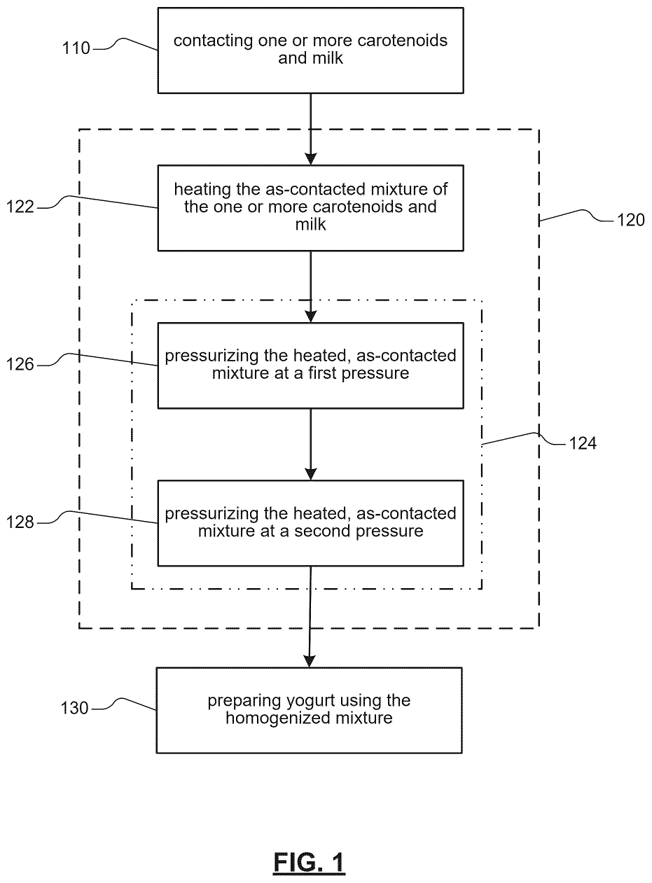

In various aspects, the present disclosure provides methods for preparing a carotenoid-fortified yogurt. For example, FIG. 1 is a flowchart illustrating an example method 100 for preparing a carotenoid-fortified yogurt. The method 100 includes contacting 110 the one or more carotenoids and milk and homogenizing 120 the as-contacted mixture. The milk may include traditional dairy milk (e.g., cow, goat) and/or any plant-based milk alternatives, where the milk has a fat content greater than or equal to about 1weight percent to less than or equal to about 5 weight percent (e.g., about 5 weight percent).

The method 100 further includes preparing 130 a yogurt using conventional processes using the homogenized mixture. Although not illustrated, it should be appreciated that, in various example embodiments, the method 100 may further include one or more cooling steps before or concurrent with the preparation 130 and/or one or more de-pressurization steps before or concurrent with the preparation 130.

In at least one example embodiment, the method 100 may include contacting 110 the one or more carotenoids and the milk at a first temperature greater than or equal to about 4° C. to less than or equal to about 20° C. In at least one example embodiment, the method 100 may include contacting 110 greater than or equal to about 1 milligrams to less than or equal to about 30 milligrams per 100 grams of milk. The one or more carotenoids may be provided as a carotenoid formulation (e.g., lutein formulation) that is dispersible in an aqueous solution and that includes a standard and an emulsifier. The carotenoid formulation should be water dispersible because its first contact with the milk to prepare the carotenoid-fortified yogurt is with the water-phase of the milk. In at least one example embodiment, the one or more carotenoids may be extracted from marigold flowers (Tagetes erecta) and standardized with sunflower oil and emulsified using a food grade emulsifier. In at least one example embodiment, the carotenoid formulation may include a lutein formulation and the lutein formulation may include Plantcol Lutein extract WS3.

The homogenization 120 of the as-contacted mixture may include heating 122 the mixture to a second temperature greater than or equal to about 55° C. to less than or equal to about 75° C. (e.g., about 65° C.) and pressurizing 124 the heated mixture. The pressurizing 124 may include a first pressurizing 126 of the heated mixture to a first pressure and a subsequent second pressurizing 128 of the heated mixture to a different second pressure that is less than the first pressure and that is also greater than atmospheric pressure. In at least one example embodiment, the pressurizing 124 may include moving the heated mixture through a pipe (or other similar structure), whereas the mixture moves from a first end of the pipe to the second end of the pipe the pressure of the mixture is lowered from the first pressure to the second pressure.

The first pressurizing 136 may include holding the first pressure for a first time period. The second pressurizing 128 may include holding the second pressure for a second time period. In at least one example embodiment, the first pressure may be greater than or equal to about 150 bar to less than or equal to about 250 bar (e.g., about 200 bar). In at least one example embodiment, the second pressure may be greater than or equal to about 40 bar to less than or equal to about 60 bar (e.g., about 50 bar).

The homogenization 120 of the as-contained mixture maximizes the mixing of the one or more carotenoids with micronized milk fat globules, and more specifically, the contact of the one or more carotenoids with milk fats of the milk such that the one or more carotenoids is protected by the milk fat from degradation. The one or more carotenoids may be embedded in milk fat globules. For example, in at least one example, embodiment, the one or more carotenoids may be encapsulated in milk fat globules.

Certain features of the current technology are further illustrated in the following non-limiting examples.

Preparation of Lutein-Fortified Yogurt Sample. A yogurt sample including 15 milligrams/100 grams of lutein, which is the maximum concentration allowed by the European Food Safety Authority (EFSA) for a dairy product, was prepared using freshly obtained whole milk, unopen Plantcol Lutein extract WS3, and a fresh commercial yogurt culture. Plantcol Lutein extract WS3 is compliant with EU regulation E 161b and is currently marketed as a natural food colorant. Plantcol Lutein extract WS3 is typically used at low dosages to provide a pale-yellow color to dairy products and beverages. Mouthfeel flavor and peach flavor were also added such that the prepared lutein-fortified yogurt sample included about 0.1 weight percent of the mouthfeel flavor and about 0.6 weight percent of the peach flavor.

Preparation of Blank (Non-Lutein-Fortified) Yogurt Sample. A blank, non-lutein-fortified yogurt sample was similarly prepared as the lutein-fortified yogurt sample using freshly obtained whole milk and a fresh commercial yogurt culture. Further, mouthfeel flavor and peach flavor were also similarly added such that the blank, non-lutein-fortified yogurt included about 0.1 weight percent of the mouthfeel flavor and about 0.6 weight percent of the peach flavor.

Compositional Analysis of the Yogurt Samples. Table 1 below shows the nutrient compositions of the as-prepared lutein-fortified yogurt sample and the as-prepared blank, non-lutein-fortified yogurt sample. There were no noted differences in the macro and micronutrient compositions between the lutein-fortified yogurt sample and the blank, non-lutein-fortified yogurt sample.

TABLE 1
Compositional Analysis of As-Prepared Yogurt Samples

| | | Lutein-Fortified |
|---|---|---|
| Components | Blank Sample | Sample |
| Ash | 0.8% | 0.8% |
| Sodium (Na) | 416 mg/kg | 427 mg/kg |
| Carbohydrates (available) | 3.6% | 3.0% |
| Total carbohydrates | 4.2% | 3.7% |
| Energy kcal (calculated) | 77 kcal/100 g | 71 kcal/100 g |
| Energy value (kJ) | 319 kJ/100 g | 295 kJ/100 g |
| Salt (NaCl) ex Na | <1 g/100 g | <1 g/100 g |
| Total sugars | 3.5% | 3.4% |
| Sucrose | <0.1% | <0.1% |
| Lactose | 2.7% | 2.6% |
| Fructose | <0.1% | <0.1% |
| Maltose | <0.1% | <0.1% |
| Glucose | <0.1% | <0.1% |
| Galactose | 0.8% | 0.8% |
| Total Solids (Milk cream, | 13.94 g/100 g | 12.99 g/100 g |
| evap) | | |
| Protein (N × 6.38) | 3.88% | 3.82% |
| Total dietary fiber | 0.6% | 0.7% |
| Total fat (Rose Gottlieb) | 5.04 g/100 g | 4.70 g/100 g |
| Trans fatty acids | 3.79 g/100 g fat | 3.92 g/100 g fat |
| Monounsaturated fatty | 1.15 g/100 g | 1.08 g/100 g |
| acids | | |
| Polyunsaturated fatty | 0.14 g/100 g | 0.15 g/100 g |
| acids | | |
| Saturated fatty acids | 3.29 g/100 g | 3.04 g/100 g |
| Trans fatty acids | 0.18 g/100 g | 0.18 g/100 g |

Microbiological and Compositional Analysis of the Yogurt Samples. Random samplings of the lutein-fortified yogurt sample and the blank, non-lutein-fortified yogurt sample underwent microbiology testing, including, for Clostridium perfringens, Coagulase positive Staphylococcus, Listeria Species, Presumptive Bacillus cereus, and Salmonella. The pathogenic microorganisms tested in the yoghurts complied with current guidelines (“Regulation (ec) no 178/2002 of the european parliament and of the council,” 2002). The sample batches were considered safe for sensory evaluation and for bioavailability testing.

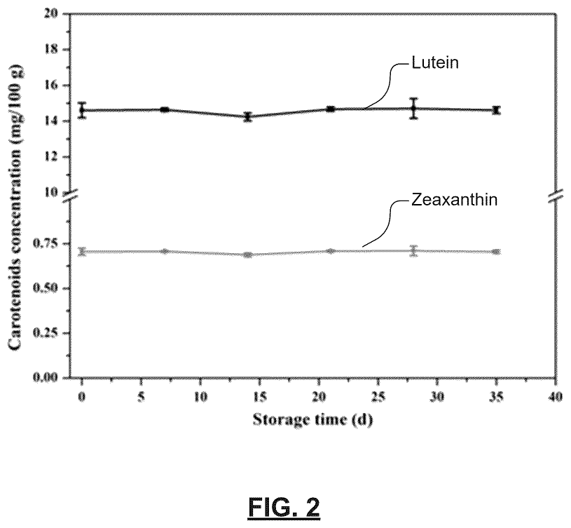

Lutein and Zeaxanthin Stability Over Shelf Life. The starting concentrations of lutein and zeaxanthin in the lutein-fortified yogurt sample were 14.60±0.41 and 0.71±0.02 mg/100 g, respectively. FIG. 2 is a graphical demonstration illustrating the concentrations of lutein and zeaxanthin in the lutein-fortified yogurt sample over a thirty-five-day period of time at 4° C. As illustrated, the concentrations of both carotenoids remained stable at day 35 (p>0.05). The stability may be influenced by the way lutein was added to the yogurt. When lutein is introduced post-fermentation via stirring, it tends to degrade in a concentration-dependent manner. Conversely, introducing lutein prior to the fermentation process yields notably enhanced storage stability. This phenomenon may be attributed to the consumption of oxygen in milk by Lactobacillus bulgaricus during fermentation.

Sensory Evaluation of Lutein-Fortified Yogurt Sample. The lutein-fortified yogurt sample and the blank, non-lutein-fortified yogurt sample underwent sensory evaluations by an expert panel of five assessors trained to international standards (ISO., 1985). The experts evaluated appearance, aroma, flavor, taste, and mouthfeel of the yogurt sample. A subsequent discussion lead by the panel leader was held between the assessors to reach a consensus on the sensory characteristics tested. Notably, the addition of sugar to the lutein-fortified yogurt sample was avoided, in order to permit a clean taste analysis.

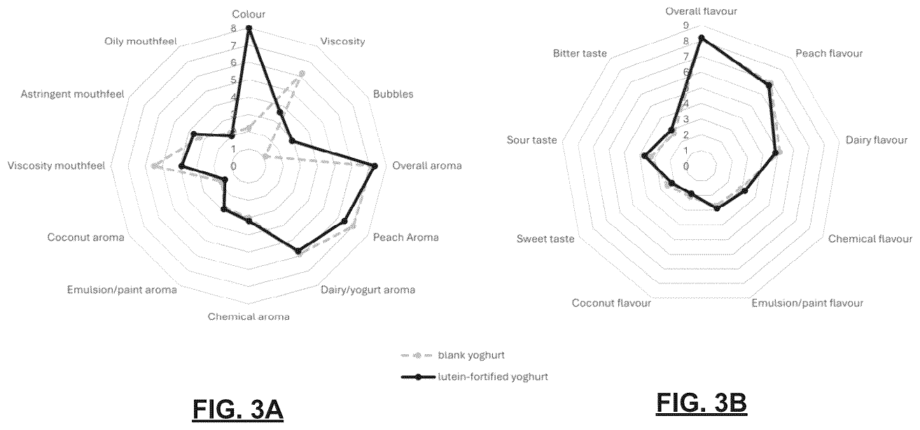

FIG. 3A is a demonstration comparing the appearance, aroma, and mouthfeel attributes of the two samples. FIG. 3B is a demonstration comparing the flavor and taste attributes of the two samples. As illustrated, there was a different in coloration between the two samples and also the lutein-fortified yogurt sample showed a lower viscosity and higher number of bubbles as compared to the blank, non-lutein-fortified yogurt sample. The flavor attributes of the lutein-fortified yoghurt sample yielded a profile very similar to that of the blank, non-lutein-fortified yogurt sample, indicating that despite the higher levels of lutein present in the lutein-fortified yoghurt sample, the mouthfeel and peach flavors added effectively masked any bitterness associated with the lutein formulations. In other words, the flavor profile of the lutein-fortified yogurt sample was comparable to that of a blank, non-lutein-fortified yogurt sample, suggesting that the mild bitterness often associated to lutein formulations can be effectively masked by flavor addition. More specifically, the lutein-associated bitterness at about 15 milligrams of lutein per 100 grams of yogurt may be masked.

Preparation of Carotenoid Extraction from Lutein Formation. To quantify an amount of lutein in the Plantcol Lutein extract WS3, three samples of the extract were prepared. The samples included 10 milligrams to 15 milligrams of the extract weighed into 50-milliliter falcon tubes and diluted with acetone until the respective measured absorbance in a spectrophotometer (Shimadzu UVmini1240, Mason Technologies, Ireland) was between about 0.2 and about 0.8. The dilution performed and its absorbance were recorded and 1 milliliter of each diluted sample was dried in a vacuum centrifuge miVac Centrifugal Concentrator (Genevac™, VWR, Ireland) at less than about 30° C. The dry residues were saponified, where 1.5 milliliters of ethanolic potassium hydroxide (5% potassium hydroxide in ethanol 96%, w: v) containing butylated hydroxytoluene (0.2% w/v) was added to the dry residues. Then, the tubes were purged with nitrogen, firmly closed, placed in the dark, and mixed for 3 hours on a shaker at 200 rotations per minute at room temperature (i.e., 20° C.-25° C.). After the incubation, 1.5 milliliters of water was added and the mixture was extracted twice with 3 milliliters of hexane. The hexane extracts were combined and dried in the vacuum centrifuge at less than 30° C. The saponified residues were resuspended in 1 milliliter of MeOH: MTBE 90:10 (v:v) and analyzed as detailed below.

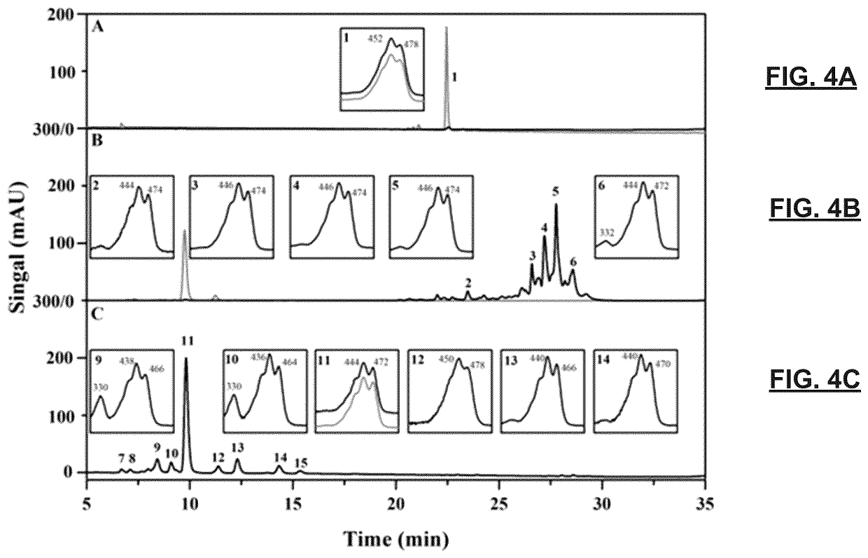

Carotenoid Profiled in Lutein Formulation and Lutein-Fortified Yogurt Sample. Plantcol Lutein extract WS3 contained 4.3±0.1 mg/g of lutein, with a carotenoid profile dominated by lutein esters. FIGS. 4A-4C show the carotenoid profile of the blank, non-lutein-fortified yogurt sample and the lutein-fortified yogurt sample. As illustrated in FIG. 4A, the blank, non-lutein-fortified yogurt sample contains only trace amounts of lutein, a small amount of β-carotene (0.051 mg/100 g), and no zeaxanthin, where Peak 1 is all-trans β-carotene and the spectrum of β-carotene standard is indicated by the grey color. While for the lutein-fortified yogurt sample without saponified treatment, as illustrated in FIG. 4B, the predominant carotenoids were lutein esters, where Peaks 2-6 are lutein and cis-lutein esters and the chromatogram of all-trans lutein standard is indicated by the grey color. Further, as illustrated in FIG. 4C, subsequent saponification of the lutein-fortified yogurt sample revealed that about 90% of carotenoids were lutein, with zeaxanthin accounting for about 4% and the other unidentified carotenoids constituting about 5.5%, where Peak 9:13-cis-lutein, Peak 10: 13′-cis-lutein, Peak 11: all-trans-lutein, Peak 12: all-trans-zeaxanthin, Peak 13:9-cis-lutein, Peak 14: 9′-cis-lutein, Peaks 7, 8, and 15: unknown, and the spectrum of all-trans lutein standard is indicated by the grey color.

Carotenoid Extraction from the Lutein-Fortified Yogurt Sample. To quantify an amount of lutein in the lutein-fortified yogurt sample, three samplings of the lutein-fortified yogurt sample were prepared. The samplings each included 100 milligrams to 150 milligrams of the lutein-fortified yogurt sample. The samplings were weighted into 50 milligrams falcon tubes and 4 milliliters of tetrahydrofuran containing 0.1% was added to each sampling. The mixtures were vortexed for 30 seconds and centrifuged at 3500 grams for 5 minute at 20° C. The upper organic phases were collected. The extraction processes were repeated until no color was observed in either the organic phase or the solid residue. After which, 10 milliliters of hexane was added to the combined of tetrahydrofuran extracts for each respective sample, followed by washing with 15 milliliters of saturated salt solution. After shaking and centrifuging, the upper organic phase was collected. The hexane extraction was repeated twice. The three extracts were then pooled and dried in the vacuum centrifuge at less than 30° C. and the dry residue was resuspended in 10 milliliters of acetone and read in the spectrophotometer. Lutein content in the lutein-fortified yogurt sample was calculated by measuring the maximum absorbance of the samplings, corrected for lutein purity. For analysis, 1 milliliter of the sample resuspended in acetone was dried in the vacuum centrifuge, resuspended in 1 milliliter of MeOH: MTBE 90:10 (v:v) and injected in the high-performance liquid chromatography.

HPLC-DAD Analysis of Carotenoids. Carotenoid profiling, relative purity and quantification were carried out in a HPLC 1260 Infinity Series (Agilent Technologies, Santa Clara, CA, USA), equipped with a diode array detector, quaternary pump, degasser, thermostatically-controlled column compartment, and a thermostatically-controlled autosampler. The diode array detector was set to 450 nanometers for zeaxanthin and 445 nanometers for lutein and-cryptoxanthin. The chromatographic separation was performed on a C30 column (250×4.6 mm i.d., 3 μm; YMC Europe, Dinslaken, Germany). A gradient program was applied with mobile phase A consisting of methanol: MTBE: water 83:15:2 (v:v), and mobile phase B consisting of methanol: MTBE: water 8:90:2 (v:v) at a flow rate of 1.0 mL·min⁻¹. The gradient initiated at 5% solvent B and increased to 20% in the first 12 minutes, to 55% over the next 8 minutes, and to 95% over the next 7 minutes. From 27 minutes to 30 minutes, solvent B was held at 95% and then resumed to the initial setting within 3 minutes. Column temperature was set at 20° C. For lutein formulation and yogurt sample analysis, 50 microliters of a sampling was injected. For carotenoid quantification in human serum, 100 microliters of a sampling were injected.

Peak identification in saponified Plantcol Lutein extract WS3 was based on retention time and UV-spectrum compared with authentic standards. The cis-isomers of lutein were identified by comparison of their respective spectra with published data. To quantify lutein extracted from the Plantcol formulation or from the yogurt sample in the spectrophotometer, the total carotenoid absorbance was corrected by the relative purity of the carotenoids present, multiplied by their respective extinction coefficient (144500 and 132900 L mol⁻¹ cm⁻¹ for lutein and zeaxanthin, respectively). For unknown peaks, the extinction coefficient of lutein was used to calculate their relative purity.

As a control expected to remain constant during the bioavailability study, a-cryptoxanthin was selected, as this carotenoid has the most similar structure to lutein. A calibration line was prepared with lutein to quantify both lutein and a-cryptoxanthin in human serum (formula y=0.07822x−0.03359). A separate calibration line was prepared to quantify zeaxanthin in human serum, with formula y=0.08528x+0.2826. Carotenoids from the single-dose bioavailability study were quantified over 4 days. For each day of analysis, a pooled serum sample prepared in-house from different subjects was analyzed in triplicate. This control analysis was carried out in order to evaluate inter-day variability (as relative standard deviation) of results obtained over the four days of analysis of the serum samples. The measured relative standard deviation for lutein quantified in the control samples over the four days in the pooled control serum was 1.1%.

Human Single-Dose Bioavailability. FortiXan was a single-dose, seventy-two hour bioavailability human trial. Ethical approval was obtained from the Research Ethics Committee, HSE, South East Technological University ethics committee (trial registration number: ISRCTN45322893). Participants were recruited via social media and email. Candidates for consideration were between the ages of 18 and 64 and were apparently in good health. Candidates were excluded if they had consumed supplements containing lutein within the three months prior to the start of the trial, were pregnant and/or breastfeeding, were taking specific prescription medications, or had allergies to any of the foods required for consumption during the trial. The candidates had a mean age of 40.40±12.66 years and a mean body mass index of 29.29±5.42. Among the participants, one was female, and four were former smokers. On average, the volunteers consumed 5.48±4.44 standard alcoholic drinks per week. Participant's demographics were collected before the start of the test. All the participants completed the study with 100% compliance.

Serum lutein and zeaxanthin concentrations were assessed in the participating over three days following the ingestion of a single-dose lutein-fortified yogurt. More specifically, on the first day of the trial, the participants (n=5) underwent arm venous blood collection on an empty stomach. Immediately after the blood draw, participants consumed a lutein-fortified yoghurt (i.e., 100 grams containing 14.7 milligrams of lutein). Breakfast (mushroom and mozzarella toasted panini) was provided two hours after yogurt ingestion, in order to minimize the potential impact of additional food ingestion on the intervention. Low-carotenoid meals were provided to the participants and the participants were asked to follow a strict diet during the four-day duration of the trial to keep lutein intake to a minimum. Blood samples were collected as indicated in Table 2 below.

TABLE 2
Blood Sampling Time and Meal Schedule in Single-
Dose Lutein-Fortified Bioavailability Study

| Withdraw | Time Point | | | |
|---|---|---|---|---|
| Number | (Hours) | Time | | Actions |
| 1 | 0 | 8 | AM | Yogurt Consumed following |
| | | | | Blood Collection |
| 2 | 1 | 9 | AM | None |
| 3 | 2 | 10 | AM | Breakfast Provided following |
| | | | | Blood Collection |
| 4 | 4 | 12 | PM | None |
| 5 | 6 | 2 | PM | Lunch Provided following Blood |
| | | | | Collection |
| 6 | 10 | 6 | PM | Dinner Provided following Blood |
| | | | | Collection |
| 7 | 24 | 8 | AM | Breakfast Provided following |
| 8 | 48 | 8 | AM | Blood Collection |
| 9 | 72 | 8 | AM | |

Fasting blood samples were collected in 8.5 milliliter vacutainer blood collection tubes containing Z Serum Sep Clot Activator (BD Vacutainer, Berkshire, UK). The blood samples were allowed to clot at room temperature (i.e., 20° C.-25° C.) for about 30 minutes and then centrifuged at 2,700 rpm for 10 minutes to separate the serum from cells. The resulting serum samples were stored at −80° C. until carotenoid analysis.

Serum was micropipetted into light-resistant 1.5 mL Eppendorf tubes labelled according to participant number. 0.3 milliliter of ethanolic butylated hydroxytoluene (250 mg/L ethanol) was added to each sample. After vortexing for 2 minutes at the highest setting, 0.5 milliliter of heptane were added to each sample. Then, the samples were vortexed at the highest setting for 2 minutes, followed by centrifugation for 5 minutes at 400 g. The upper heptane layer, of approximately 0.6 milliliter, was removed to a new light-resistant Eppendorf tube and the heptane extraction was repeated. The combined extracts were dried in a vacuum centrifuge at less than 30° C. and resuspended in 0.2 milliliter of methanol: MTBE 90:10 (v:v). The 0.2 milliliters samples were vortexed at the lowest setting for 1 minutes to resuspend the dry residue and pipetted into 2.5 milliliter vials containing 0.35 milliliter glass inserts. High-performance liquid chromatography analysis was then performed, as discussed above.

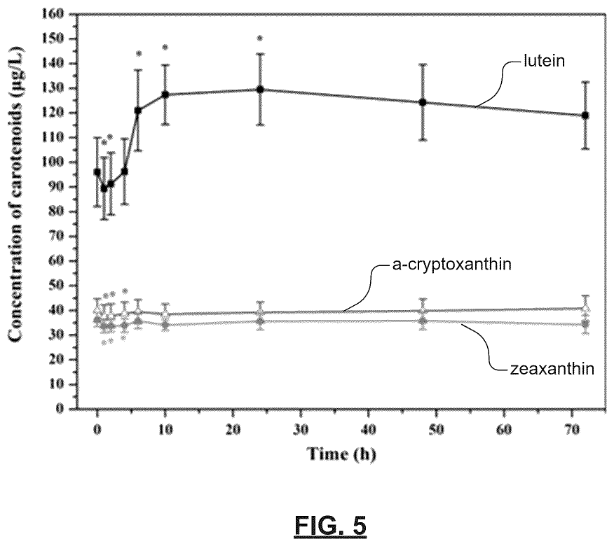

FIG. 5 is a graphical demonstration illustrating the concentrations of lutein, a-cryptoxanthin, and zeaxanthin in serum collected from the participants. Serum concentrations of all three carotenoids showed a significant decrease (p<0.05) in the initial 4 hours of the trial compared to baseline concentrations. The cause of this decrease might be the 300 milliliters of water provided to the participants to drink immediately after the first blood draw. One pharmacokinetic analysis of deuterium-labelled water has shown that ingested water appears in plasma and blood cells within five minutes of ingestion and is completely absorbed in approximately seventy-five to one-hundred and twenty minutes. Therefore, it is likely that the water drank by the participants caused plasma dilution and thus decreased the initial serum carotenoid concentration. Lutein concentration in serum increased 4 hours after the ingestion of the lutein-fortified yogurt sample, and the highest serum lutein concentrations were detected at 24 hours. The concentration of a-cryptoxanthin, used as a control carotenoid was expected to remain constant over the trial and in fact showed no significant changes in concentration from 4 hours until the end of the trial (39.54±0.38 μg/L). Zeaxanthin showed a similar behavior to a-cryptoxanthin and remained stable at 35.09±0.38 μg/L.

The pharmacokinetic parameters of lutein and zeaxanthin are shown in Table 3 below. As noted, serum lutein Cₘₐₓ was significantly higher (38.39 μg/L, p<0.05) than baseline, with an AUC₀₋₇₂ of 1946.86 h μg L⁻¹. However, no significant changes were observed in the Cₘₐₓ or AUC of zeaxanthin.

TABLE 3
Lutein and Zeaxanthin Pharmacokinetic Parameters After
a Single-Dose of the Lutein-Fortified Yogurt Sample

| | Lutein | Zeaxanthin |
|---|---|---|
| Dose (mg) | 14.61 ± 0.41 | 0.65 ± 0.02 |
| Baseline (μg/L) | 95.99 ± 31.17 | 36.19 ± 6.26 |
| Cₘₐₓ (μg/L) | 134.38 ± 26.94 | 37.96 ± 6.99 |
| Sig. | 0.015 | 0.076 |
| Tₘₐₓ (h) | 14.00 ± 9.27 | 40.40 ± 24.02 |
| AUC_{(0-72 h)} (h · μg/L) | 8841.31 ± 2220.18 | 2530.55 ± 512.66 |
| ΔC (μg/L) | 38.39 ± 22.41 | — |
| | \| 2.63 ^{a} | |
| ΔAUC₀₋₇₂ (h · μg/L) | 1946.86 ± 1514.72 | — |
| | \| 133.35 ^{a} | |

In Table 3, values are presented as means±SD, ^{a} values are standardized per milligram of each carotenoid, the difference from baseline were made using t-tests, and-data not available)

Statistical analysis was performed using OriginPro 8.5 (OriginLab, USA) and SPSS statistics 22 (IBM, USA). Paired t-tests were used to analyze statistical significance of the observed changes in lutein stability in the lutein-fortified yogurt sample. Statistical significance was set at p<0.05. The primary endpoints studied for lutein bioavailability were the seventy-two-hour area under the curve for serum lutein (AUC₀₋₇₂ₕ), calculated using the linear trapezoidal rule, and the maximum concentration (Cₘₐₓ ), determined as the time at which the maximum concentration of serum lutein was observed (tₘₐₓ). To meet the assumption of normality, statistics on AUC₀₋₇₂ₕ and Cₘₐₓ were based on log transformed values for individual subjects. Differences in serum carotenoid concentration at each time point were assessed by paired t-test.

The absorption rate per unit mass (ARUM) of lutein, calculated as the ratio of the maximum ΔC of lutein in serum/plasma to the amount of lutein ingested, was used to compare the bioavailability of lutein from the lutein-fortified yoghurt with that from foodstuffs and dietary supplements from previously recorded studies. As summarized in Table 4 below, ARUM for the lutein-fortified yogurt sample was 2.6 μg/L per mg of lutein. ARUM was also calculated for foodstuffs and dietary supplements studied previously. ARUM for the as-prepared lutein-fortified yogurt sample was similar to the lutein-fortified yogurt sample of lutein-fermented milk. When compared with foods with naturally occurring lutein, their ARUM were superior (5.0 for broccoli and 7.3-10.5 for spinach). ARUM in dietary supplements showed a high variability, with lutein oil suspensions showing an ARUM of 28.5, 25.9, or 5.3. Lutein dietary supplements prepared with non-oil based formulations generally showed a lower ARUM. For instance, two beadlet formulations showed an ARUM of 1.5 and 7.8, respectively, whereas lutein powder formulations (unprotected lutein microcrystals) showed an ARUM of 6-8.

TABLE 4
Single-Dose Bioavailability Study of Lutein From Different Sources

| | | Lutein | | | Tₘₐₓ | ΔC | |
|---|---|---|---|---|---|---|---|
| Type | Food | (mg) | Fat | | (h) | (μg/L) | ARUM |
| Functional | Lutein-Fortified | 14.6 | 4.7 | g | 14.0 ± 9.3 | 38.4 ± 22.4 | 2.6 |
| foods | Yogurt | | | | | | |
| | Lutein-Fortified | 8 | >13 | g | 6.5 | 21.6 | 2.7 |
| | Fermented Milk | | | | | | |
| | Lutein-Fortified | 16 | >13 | g | 6.5 | 39.3 | 2.5 |
| | Fermented Milk | | | | | | |
| Foods | Broccoli | 8 | 10 | g | 10 | 39.8 | 5.0 |
| | (cooked) | | | | | | |
| | Spinach | 9.2 | 10 | g | 10 | 96.7 | 10.5 |
| | (cooked) | | | | | | |
| | Spinach | 6 | ~20 | g | 24 | 43.5 | 7.3 |
| | (cooked) | | | | | | |
| | Egg (cooked) | 6 | ~20 | g | 24 | 72.8 | 12.1 |
| | Chlorella | 7.1 | 1.4 | g | 19.0 ± 15.7 ^{a} | 20.4 ^{b} | 2.9 ^{b} |
| | vulgaris | | | | | | |
| | (powder) | | | | | | |
| Dietary | Oil suspension | 10 | 57% | | 20 ^{a} | 284.5 ± 208.0 | 28.5 |
| supplements | Non-disclosed | 10 | 57% | | 16 ^{a} | 112.5 ± 75.7 | 11.3 |
| | Oil suspension | 9 | 10.3 | g | 12 | 233.2 | 25.9 |
| | FloraGLO ® | 20.4 | nd | | 17.4 ± 7.5 | 158.7 ± 74.0 | 7.8 |
| | Lutein beadlet | | | | | | |
| | Lyc-O-Lutein | 20.9 | nd | | 19.8 ± 21.1 | 31.3 ± 30.7 | 1.5 |
| | beadlet | | | | | | |
| | Powder | 10 | nd | | 12 | 80 | 8 |
| | Powder | 20 | nd | | 12 | 120 | 6 |
| | Powder | 40 | nd | | 12 | 250 | 6.3 |
| | Oil suspension | 20 | nd | | 12 | 120 | 6 |
| | with n-3 fatty | | | | | | |
| | acids | | | | | | |
| | Oil suspension | 6 | ~20 | g | 24 | 31.7 | 5.3 |
| | Oil suspension (lutein esters) | 5.5 | ~20 | g | 24 | 37.8 | 6.9 |

In Table 4, values are presented as means±SD, ^{a} Tₘₐₓ is estimated according to standard methods for assessing drug pharmacokinetics, b Data displayed is geometric mean; ΔC represents the baseline-corrected lutein Cₘₐₓ; ARUM is the absorption rate per unit mass of lutein, calculated as the ratio between ΔC of lutein and the amounts of lutein intake (μg/L per mg of lutein intake), and Nd referents to those items non disclosed by the as-relied on publication.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A carotenoid-fortified yogurt comprising:
greater than or equal to about 0.001 weight percent to less than or equal to about 0.03 weight percent of one or more carotenoids.

2. The carotenoid-fortified yogurt of claim 1, wherein the one or more carotenoids include lutein, zeaxanthin, astaxanthin, lycopene, or any combination thereof.

3. The carotenoid-fortified yogurt of claim 1, wherein the one or more carotenoids include lutein.

4. The carotenoid-fortified yogurt of claim 1, wherein the carotenoid-fortified yogurt includes about 0.5 weight precent of the one or more carotenoids.

5. The carotenoid-fortified yogurt of claim 1, wherein the carotenoid-fortified yogurt includes greater than or equal to about 1 milligrams to less than or equal to about 30 milligrams of the one or more carotenoids per 100 grams of yogurt.

6. The carotenoid-fortified yogurt of claim 5, wherein the carotenoid-fortified yogurt includes about 15 milligrams of the one or more carotenoids per 100 grams of yogurt.

7. The carotenoid-fortified yogurt of claim 1, wherein the carotenoid-fortified yogurt further comprises:
a mouthfeel flavor.

8. The carotenoid-fortified yogurt of claim 7, wherein the mouthfeel flavor has an expressed color similar to that of the one or more carotenoids.

9. The carotenoid-fortified yogurt of claim 7, wherein the one or more carotenoids include lutein and the mouthfeel flavor includes peach, banana, or a combination thereof.

10. A method for preparing a carotenoid-fortified yogurt, the method comprising:
pressurizing a mixture including one or more carotenoids and milk at a first pressure greater than or equal to about 150 bar to less than or equal to about 250 bar; and
after the pressurizing of the mixture at the first pressure, pressurizing the mixture at a second pressure greater than or equal to about 40 bar to less than or equal to about 60 bar.

11. The method of claim 10, wherein the mixture includes greater than or equal to about 0.001 weight precent to less than or equal to about 0.03 weight percent of the one or more carotenoids.

12. The method of claim 10, wherein the first pressure is about 200 bar and the second pressure is about 50 bar.

13. The method of claim 10, wherein the method further comprises:
depressurizing the mixture from the first pressure to the second pressure.

14. The method of claim 10, wherein the method further comprises:
before the pressurizing of the mixture at the first pressure, heating the mixture to a temperature greater than or equal to about 55° C. to less than or equal to about 75° C.

15. The method of claim 14, wherein the temperature is about 65° C.

16. The method of claim 10, wherein the method further comprises:
preparing the mixture by contacting the one or more carotenoids and milk at a temperature greater than or equal to about 4° C. to less than or equal to about 20° C.

17. A method for preparing a carotenoid-fortified yogurt, the method comprising:
contacting one or more one or more carotenoids and a milk to prepare a mixture;
heating the mixture to a temperature greater than or equal to about 4° C. to less than or equal to about 20° C.;
pressurizing the heated mixture at a first pressure greater than or equal to about 150 bar to less than or equal to about 250 bar; and
after the pressurizing of the mixture at the first pressure, pressurizing the heated mixture at a second pressure greater than or equal to about 40 bar to less than or equal to about 60 bar.

18. The method of claim 17, wherein the mixture includes greater than or equal to about 0.001 weight percent to less than or equal to about 0.03 weight percent of the one or more carotenoids.

19. The method of claim 17, wherein the method further comprises:
depressurizing the mixture from the first pressure to the second pressure.

20. The method of claim 17, wherein the one or more carotenoids include lutein.
